(19) United States
(12) Patent Application Publication
(10) Pub. No.: US 2026/0240412 A1
(43) Pub. Date: Aug. 20, 2026

(54) VALVE FOR A MEDICAL DEVICE

(71) Applicant: Boston Scientific Scimed, Inc., Maple Grove, MN (US)

(72) Inventors: Jenny Dandin, Worcester, MA (US); Henry Stock, Boston, MA (US)

(73) Assignee: Boston Scientific Scimed, Inc., Maple Grove, MN (US)

(21) Appl. No.: 19/541,012

(22) Filed: Feb. 16, 2026

Related U.S. Application Data
(60) Provisional application No. 63/759,328, filed on Feb. 17, 2025.

(51) Int. Cl. A61B 1/00

(52) U.S. Cl. CPC A61B 1/00068; A61B 1/0011

(57) ABSTRACT

Devices, systems, and methods for a valve assembly for a medical device. The valve assembly may include a valve stem and a first seal body. The valve stem may include a proximal end, a distal end, an outer surface, a first orifice, a second orifice distal of the first orifice, and a lumen in fluid communication with the first orifice and the second orifice. The first seal body may extend along the outer surface of the valve stem. The first seal body may include an opening aligned with the first orifice, a first seal proximal of the opening and a second seal distal of the opening. The valve assembly may include a second seal body along the outer surface of the valve stem and spaced from the first seal body. The second seal body may include an opening aligned with the third orifice.

### CROSS-REFERENCE TO RELATED APPLICATIONS

This application claims the benefit of U.S. Provisional Patent Application Serial No. 63/759,328 filed on February 17, 2025, the disclosure of which is incorporated herein by reference.

### TECHNICAL FIELD

This disclosure relates generally to valve assemblies and methods, and particularly to valves for medical devices.

### BACKGROUND

A wide variety of intracorporeal medical devices and systems have been developed for medical use, for example, for endoscopic procedures. Some of these devices and systems include guidewires, catheters, catheter systems, endoscopic instruments, valves, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In an example, a valve for a medical device may include a valve stem including a proximal end, a distal end, an outer surface, a first orifice, a second orifice distal of the first orifice, and a lumen in fluid communication with the first orifice and the second orifice, and a first seal body extending along the outer surface of the valve stem, the first seal body having an opening aligned with the first orifice, a first seal proximal of the opening, and a second seal distal of the opening.

Alternatively or additionally to any of the examples above, the valve stem and the first seal body may be formed from one or more polymers.

Alternatively or additionally to any of the examples above, the first seal may have a first maximum outer diameter and the second seal may have a second maximum outer diameter that is less than the first maximum outer diameter.

Alternatively or additionally to any of the examples above, the first seal body may include a third seal distal of the second seal.

Alternatively or additionally to any of the examples above, the first seal may have a first maximum outer diameter, the second seal may have a second maximum outer diameter, and the third seal may have a third maximum outer diameter that is less than one or both of the first maximum outer diameter and the second maximum outer diameter.

Alternatively or additionally to any of the examples above, the valve may include a second seal body extending along the outer surface of the valve stem spaced from and distal of the first seal body.

Alternatively or additionally to any of the examples above, the second seal body may terminate proximal of the distal end of the valve stem.

Alternatively or additionally to any of the examples above, the second seal body may include an opening aligned with the second orifice.

Alternatively or additionally to any of the examples above, the second seal body may include a first seal, a second seal distal of the first seal and proximal of the opening of the second seal body, and a third seal distal of the opening of the second seal body.

Alternatively or additionally to any of the examples above, the second seal body may include a first seal having a first maximum outer diameter, a second seal distal of the first seal and having a second maximum outer diameter, and a third seal distal of the second seal and having a third maximum outer diameter, wherein the first maximum outer diameter may be greater than one or both of the second maximum outer diameter and the third maximum outer diameter.

Alternatively or additionally to any of the examples above, the second maximum outer diameter and the third maximum outer diameter may be equal.

In another example, a valve for a medical may include an interface member, a valve stem configured to couple with the interface member, including a proximal end, a distal end, an outer surface, a first orifice, a second orifice distal of the first orifice, and a lumen in fluid communication with the first orifice and the second orifice, a first seal body extending along the outer surface of the valve stem, the first seal body having an opening aligned with the first orifice and a plurality of seals, and a second seal body extending along the outer surface of the valve stem and spaced from the first seal body, the first seal body having an opening aligned with the second orifice and a plurality of seals.

Alternatively or additionally to any of the examples above, the proximal end of the valve stem may be open to the lumen and the distal end of the valve stem is closed to the lumen.

Alternatively or additionally to any of the examples above, the interface member may be configured to extend through the open proximal end and into the lumen of the valve stem.

Alternatively or additionally to any of the examples above, the plurality of seals of the first seal body may include at least two seals distal of the opening of the first seal body and the plurality of seals of the second seal body may include at least two seals proximal of the opening of the second seal body.

Alternatively or additionally to any of the examples above, the valve stem may include a third orifice in fluid communication with the lumen and located between the first orifice and the second orifice.

Alternatively or additionally to any of the examples above, the valve stem may include a third orifice in fluid communication with the lumen and located between and spaced from the first seal body and the second seal body.

In another example, a method of manufacturing a valve for a medical device may include forming a valve stem with a first molding shot, the valve stem including a proximal end, a distal end, an outer surface, a first orifice, a second orifice distal of the first orifice, and a lumen in fluid communication with the first orifice and the second orifice, and forming a first seal body and a second seal body spaced from the first seal body on the outer surface of the valve stem with a second molding shot.

Alternatively or additionally to any of the examples above, the method may include sealing a proximal opening of the valve stem with an interface member.

Alternatively or additionally to any of the examples above, the first seal body may include an opening aligned with the first orifice and a plurality of seals and the second seal body may include an opening aligned with the second orifice and a plurality of seals.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic perspective view of an illustrative valve;

FIG. 4 depicts a schematic cross-section view of the illustrative valve depicted in FIG. 3;

FIG. 5 depicts a schematic perspective view of an illustrative valve stem assembly;

FIG. 6 depicts a schematic cross-section view of the illustrative valve stem assembly depicted in FIG. 5;

FIG. 7 depicts a schematic perspective view of an illustrative valve in a valve well;

FIG. 8A depicts a schematic cross-section view of the illustrative valve in the valve well of FIG. 7, with the valve in a first configuration;

FIG. 8B depicts a schematic cross-section view of the illustrative valve in the valve well of FIG. 7, with the valve in a second configuration;

FIG. 9 is a schematic box diagram of an illustrative method of forming a valve stem assembly; and

FIGS. 10A-10C are schematic diagrams of assembling a valve.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. The concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, the particular feature, structure, or characteristic may be effected in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s).

For the purpose of clarity, certain identifying numerical nomenclature (e.g., first, second, third, fourth, etc.) may be used throughout the description and/or claims to name and/or differentiate between various described and/or claimed features. The numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element.

The detailed description is intended to illustrate but not limit the disclosure. The various elements described may be arranged in various combinations and configurations without departing from the scope of the disclosure. The detailed description illustrates example configurations of the disclosure.

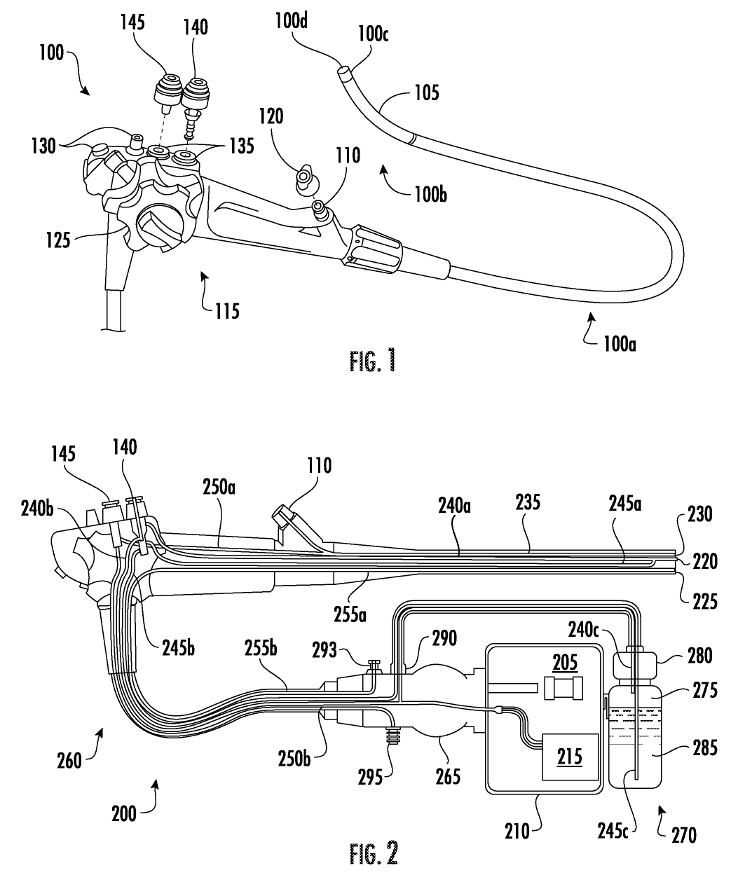

With reference to FIG. 1, an illustrative endoscope 100 is schematically depicted and FIG. 2 schematically depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump (e.g., an air pump 215), such as an air feed pump, in the unit 210.

The endoscope shaft 100a may include a distal tip 100c (e.g., a distal tip unit adapted to be inserted into a body cavity of a patient) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob controls up-down steering and another knob controls left-right steering). One or more (e.g., one, two, a plurality, etc.) of video switches 130 for remotely operating the video processing unit 210 may be arranged on a proximal end side of the handle 115.

The handle 115 may be provided with dual valve locations 135. One of the valve locations 135 may have or receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220, as depicted in FIG. 2.

The other valve location 135 may have or receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water container or reservoir 270 (e.g., a water bottle and/or other suitable reservoir or container) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250bis fluidly connected to the valve location 135 for the suction valve 145 and configured such that operation of the suction valve 145 in the well controls suction applied to the working channel 235 of the endoscope 100.

Referring to FIG. 2, an illustrative operation of an endoscope system 200, including an endoscope such as the endoscope 100 depicted in FIG. 1, is explained. Air from the air pump 215 in the video processing unit 210 may flow through the connector portion 265 and branch to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user’s finger on the valve, air is allowed to flow out of the valve 140 to atmosphere. In a first position, the user’s finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve 140, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some configurations, outflow check valves or other one-way valve configurations (not shown) may be placed in the path of the lens wash supply tubing to help prevent water from back-flowing into the reservoir 270 after the water has passed the valve.

A relatively higher flow rate compared to lens wash is typically required for irrigation water, since a primary use is to clear the treatment area in the patient of debris that obstructs the user’s field of view. Irrigation is typically achieved with the use of a pump (e.g., peristaltic pump), as described. In configurations with an independent water source for irrigation, tubing placed in the bottom of a water source may be passed through the top of the water source and threaded through the head on the upstream side of the pump. Tubing on the downstream side of the pump is connected to the irrigation feed line 255b in the umbilical 260 and the irrigation supply line 255a of endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical 260, and down the irrigation supply line in the shaft 100a of the endoscope 100 to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash tubing 245c, may be placed in the path of the irrigation supply tubing to help prevent back-flow into the reservoir after water has passed the valve.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve 145 may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilical 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position)

A medical cleaning valve (or cleaning valve or cleaning adapter) may be configured to provide cleaning functionality to fluid (e.g., air and water) channels of the endoscope 100. In some examples, the cleaning valve may be configured for use in one or more precleaning steps of reprocessing the endoscope 100 after use. In a first configuration, the cleaning valve may provide a continuous feed of gas (e.g., air or carbon dioxide) to both air and water channels (e.g., the gas supply line 240a and the lens wash supply line 245a, respectively) in a handle and/or shaft of the endoscope 100, and through an air/water nozzle (e.g., the gas/lens wash nozzle 220) at the distal end of the endoscope 100. In a second configuration, the cleaning valve may feed water into the air channel in the handle and shaft of the endoscope 100, and through the air nozzle at the distal end of the endoscope 100.

One or more of the valve configurations described herein may include a cleaning valve (or other suitable valve) that is appropriate for single-use and therefore may be disposable. Accordingly, the valve may be made from a limited number of parts and materials, e.g., to limit its cost and/or manufacturing complexity. For example, multiple seals may be integrally formed with a valve stem. In another example, the valve may have an interface member (e.g., a cap), which may combine and simplify the functionality of a number of components, such as by connecting the valve stem to a valve well, sealing an opening to a lumen in the valve stem, and/or biasing the valve stem into a position relative to the valve well. In yet another example, the valve stem may be formed with a closed distal end. In some examples, the cleaning valve may be configured for use in the gas/lens wash valve well of the endoscope 100.

Enabling the disposability of cleaning valves in an economically viable manner can lead to safer cleaning valves that may minimize or prevent the spread of infection by eliminating or reducing opportunities for germs to be inadvertently introduced into a patient such as due to improper cleaning of a reusable cleaning valve. Further, removing the need of reprocessing cleaning valves between uses can reduce or eliminate staff, tracking procedures, verification procedures, processing equipment, time, and/or money needed to perform reprocessing operations.

A valve configured for use in the gas/lens wash valve well of the endoscope 100 may have any suitable configuration. In some examples, the valve configured for use in the gas/lens wash valve well of the endoscope 100 may be configured as a cleaning valve or adapter and may include a valve shaft assembly having a polymer shaft, one or more seals formed from a polymer and configured to aid in directing fluid through different flow paths within the gas/lens wash valve well of the endoscope 100, and an interface member formed from a polymer material. In one example, the valve shaft and the one or more seals of the valve shaft assembly may be formed by injection molding the valve shaft and over-molding the one or more seals over the valve shaft. The interface member may be coupled with the valve shaft and if the valve shaft includes a vent opening, the interface member coupled with the valve shaft may block the vent opening. In some examples, the blocking of the vent hole through a valve shaft may facilitate differentiating between a cleaning valve and a gas/lens wash valve, as the cleaning valve is configured for use in the same valve well as the gas/lens wash valve. Although the valve configurations discussed herein may be described as being configured for use as a cleaning valve configured to facilitate cleaning the endoscope 100, the valve configurations may be used for other suitable purposes.

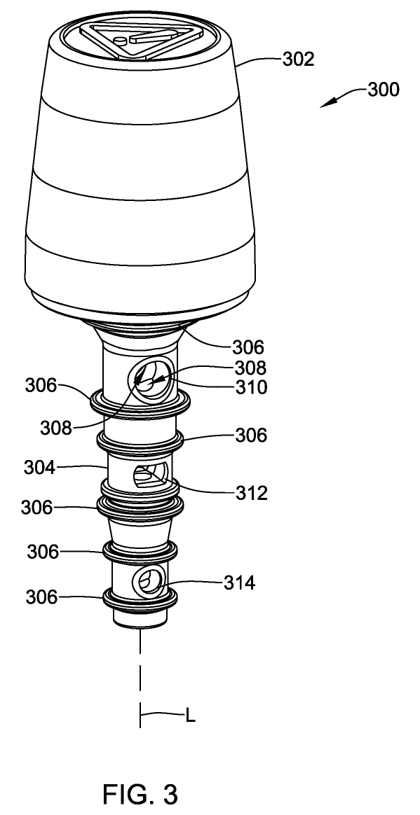

FIG. 3 schematically depicts a perspective view of an illustrative configuration of a valve assembly or valve 300 (hereinafter “valve”). In some examples, the valve 300 may be a cleaning valve used in cleaning/reprocessing of endoscopes and/or associated equipment, such as at the end of a procedure on a patient. The configurations of the valve 300 described herein are not limited in this context.

The valve 300 may include an interface member 302, a valve stem 304, one or more seals 306, and/or one or more other suitable components or features. As depicted in FIG. 3, the valve stem 304 may extend along a longitudinal axis L and the one or more seals 306 may extend around the longitudinal axis L. The valve stem 304 may include a lumen 308 and one or more orifices in fluid communication with the lumen 308. In some example configurations of the valve stem 304, the valve stem 304 may include a first orifice 310, a second orifice 312, and a third orifice 314, all of which may be in fluid communication with the lumen 308. In some examples, one or more of the first orifice 310, the second orifice 312, and the third orifice 314 may be transverse to (e.g., perpendicular to or at one or more other crossing angle with) the lumen 308 and/or the longitudinal axis L. Although only a single lumen 308 is depicted in the Figures, the valve stem 304 may include a plurality of lumens.

The interface member 302 may include one or more indications the valve 300 is a cleaning valve and is not a gas/lens wash valve. In one example, a proximal facing surface of the interface member 302 may include indicia indicating the valve 300 is a cleaning valve and is not a gas/lens wash valve. In some examples, the indicia may include a geometric shape, wording, raised surface, and/or a symbol. In one example, the proximal facing surface of the interface member 302 may include raised surfaces having the form of an exclamation point symbol within a triangular geometric shape, as depicted in FIG. 3, but other suitable indicia are contemplated.

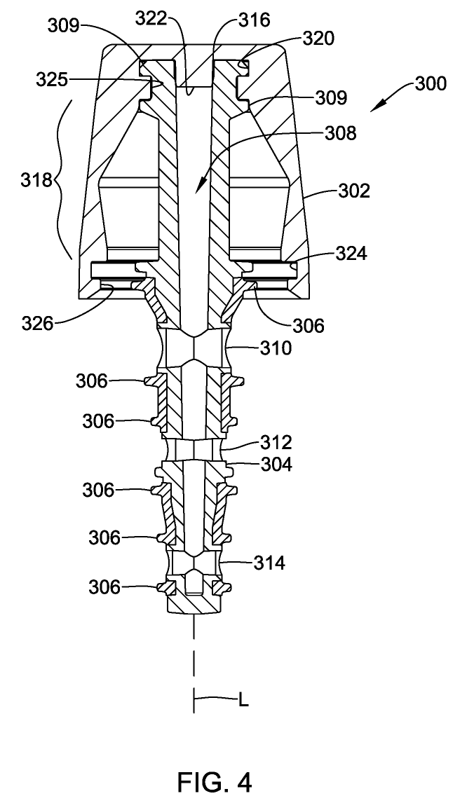

FIG. 4 depicts a schematic cross-section view of the valve 300 depicted in FIG. 3. In some examples, the valve stem 304 of the valve 300 may include a fourth orifice 316 at a proximal end of the valve stem 304 and/or the lumen 308, which may be considered a vent opening. The valve stem 304 may include one or more fewer, additional, and/or alternative orifices than the four orifices 310, 312, 314, 316 depicted in FIG. 4. In some examples, the valve stem 304 may include one or more seating members 309 configured to engage the interface member 302, one or more centering members 307, and/or other suitable features.

The orifices 310, 312, 314, 316 of the valve stem 304 may include one or more axial and/or one or more radial holes or openings. For example, and as depicted in FIG. 4, the fourth orifice 316 may comprise an axial hole and each of the first orifice 310, the second orifice 312, and the third orifice 314 may comprise respective radial through-holes spaced axially from one another along the longitudinal axis L of the valve stem 304. Although the radial orifices are depicted as extending through an outer diameter of the valve stem 304 and at the same or similar circumferential locations, one or more of the orifices may not extend entirely through the diameter of the valve stem 304 and/or may be located at other suitable radial locations relative to at least one other orifice. In some examples, more than three or less than three radial orifices may be utilized. In some examples, the radial orifices 310, 312, 314 may allow fluid to pass from the outside diameter of the valve stem 304 to the lumen 308 defined by an inner diameter of the valve stem 304 and out one or more other orifices of the valve stem 304.

As depicted in FIG. 4, the interface member 302 may include one or more spring portions 318, one or more stem recesses 320, one or more stem plugs 322, one or more well recesses 324, one or more stem protrusions 325, one or more well retention members 326, and/or one or more other suitable components or features configured to engage with the valve stem 304 and/or a valve well. When included, the one or more stem plugs 322 of interface member 302 may be inserted into the fourth orifice 316 to seal the fourth orifice 316 and prevent fluid from escaping the valve 300 via the fourth orifice 316. In some examples, the interface member 302 may include a cap and/or other suitable components. One or more aspects of the interface member 302 may be formed separate from or as part of the valve stem 304.

The spring portion 318 of the interface member 302 may be used to facilitate transitioning the valve 300 from a first configuration to a second configuration when installed into a valve well. For example, the first configuration may be a standby state and the second configuration may be a flushing state. In some embodiments, the spring portion 318 may bias the valve 300 into one of the first and second configurations. For example, the spring portion 318 may bias the valve 300 into the standby state and the valve 300 may be transitioned into the flushing state by depressing the interface member 302 in a distal direction. Any suitable biasing material or biasing mechanism may be incorporated into the spring portion 318 of the interface member 302 to bias the valve 300 into one of the first and second configurations.

The seating members 309 of the valve stem 304 may comprise circumferential protrusions that define a channel therebetween. When assembled, the seating members 309 may fit, at least partially, into the stem recesses 320 of the interface member 302, respectively. Further, the stem protrusion 325 of the interface member 302 may fit, at least partially, into the channel defined between seating members 309 of the valve stem 304. In some examples, the interface member 302 and valve stem 304 may be attached together via a snap fit. In some examples, the snap fit may be a snap interference fit. It is understood that the proximal end of the valve stem 304 and the interface member 302 may have any interlocking or mating features (e.g., protrusions, recesses, threads, or the like) for attachment to one another. One or more other suitable techniques for attaching the interface member 302 to the valve stem 304 may be utilized.

The well recess 324 and the retention member 326 of the interface member 302 may be used to connect the valve 300 to a valve well via a corresponding circumferential protrusion of the valve well (e.g., a connection flange, not depicted in FIG. 4). The connection between the interface member 302 and the valve well 350 may be a snap fit, a snap interference fit, and/or a threaded fit. One or more other suitable techniques for attaching the valve 300 to a valve well may be utilized.

Any suitable materials may be utilized to form the valve 300 or components thereof. For example, suitable materials for forming one or more components of the valve 300 include, but are not limited to, metal materials, polymer materials, plastic materials, bioplastic materials, recyclable materials, thermoplastic materials, lubricious materials, flexible materials, thermoplastic elastomers (TPE), thermoplastic polyurethane (TPU), polycarbonate, acrylonitrile butadiene styrene (ABS), high-density polyethylene (HDPE), nylon, polyether ether ketone (PEEK), silicone, liquid silicone rubber (LSR), steel, aluminum, and/or other suitable material. In one example, the valve stem 304 may be formed from a plastic material, the seals 306 may be formed from a silicone, and the interface member 302 may be formed from a silicone. Other suitable materials may be utilized to form the valve 300.

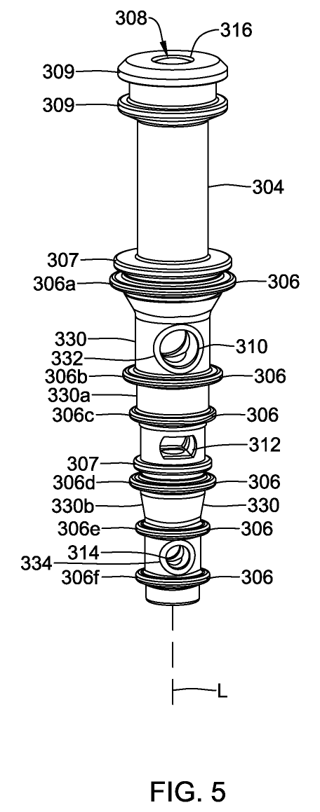

FIG. 5 schematically depicts a perspective view of an illustrative configuration of a valve stem assembly comprising the valve stem 304 and the seals 306 along an outer surface or perimeter of the valve stem 304. The valve stem 304 may include a proximal end proximate the fourth orifice 316, a distal end distal of the third orifice 314, an outer surface, and/or one or more other suitable features or components. As discussed, the valve stem 304 may define or otherwise include the lumen 308 and the one or more orifices 310, 312, 314, 316.

The seals 306 have any suitable form configured to facilitate controlling flow through a valve well. In some examples, the seals 306 may be protrusions extending radially outward from the longitudinal axis L and circumferentially around the valve stem 304, but other suitable configurations are contemplated. The seals 306 may be any suitable type of seal. For example, the seal 306 may have a one-wave valve or seal configuration, a wiper blade seal configuration, and/or other suitable type of seal configuration. In one example, one or more of the seals 306 may be configured as a wiper blade seal extending circumferentially around the valve stem 304.

In some examples, the seals 306 may be formed as part of a seal body 330. The seal body 330 may have any suitable form. In some examples, the seal body 330 may be elongated and may have an inner surface configured to extend along an outer surface or outer perimeter of the valve stem 304. As depicted in FIG. 5, the valve stem assembly may include a first seal body 330a with one or more seals 306 and a second seal body 330b with one or more seals 306. In some examples, the second seal body 330b may be spaced from and distal of the first seal body 330a, but other suitable configurations are contemplated. Although two seal bodies 330 are depicted in FIG. 5, more than two seal bodies 330 or a single seal body 330 may be utilized for providing the seals 306 around and/or along the valve stem 304.

The first seal body 330a may have any suitable configuration. In some examples, the first seal body 330a may be elongated and include one or more first openings 332 aligned with or configured to be aligned with the first orifice 310 of the valve stem 304, as depicted in FIG. 5. In some examples, the first seal body 330a may include a plurality of seals 306. In one example, the first seal body 330a may include a first seal 306a at a location proximal of the first opening 332 and a second seal 306b distal of the first opening 332. In some examples, the first seal body 330a may include one or more additional and/or alternative seals 306 including, for example, a third seal 306c at a location distal of the second seal 306b. In some examples, the first seal 306a, the second seal 306b, and the third seal 306c may be located on the first seal body 330a and/or the valve stem 304 at a location proximal of the second orifice 312.

The second seal body 330b may have any suitable configuration. In some examples, the second seal body 330b may be elongated and include one or more second openings 334 aligned with or configured to be aligned with another orifice (e.g., the third orifice 314) of the valve stem 304, as depicted in FIG. 5. In some examples, the second seal body 330bmay include a plurality of seals 306. In one example, the second seal body 330b may include a fourth seal 306d at a location proximal of the second opening 334, a fifth seal 306e distal of the fourth seal 306d and proximal of the second opening 334, and a sixth seal 306f distal of the second opening 334, but the second seal body 330b may have other suitable configurations of the seals 306. In some examples, the fourth seal 306d, the fifth seal 306e, and the sixth seal 306f may be located on the second seal body 330b and/or the valve stem 304 at a location distal of the second orifice 312.

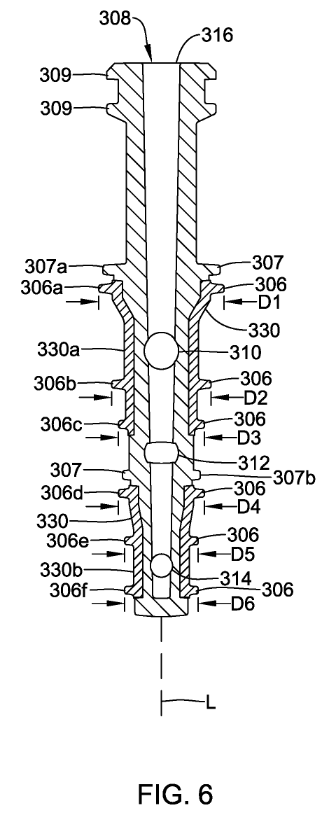

FIG. 6 depicts a schematic cross-section view of the valve stem assembly depicted in FIG. 5, comprising the valve stem 304 and the seals 306 extending along an outer surface of the valve stem 304 including the lumen 308. The lumen 308 may be, may be defined by, and/or may include a channel placing the one or more of the orifices 310, 312, 314, 316 in fluid communication with one another. In the configuration depicted in FIG. 6, the lumen 308 may place the first orifice 310, the second orifice 312, the third orifice 314, and the fourth orifice 316 in fluid communication with one another. As discussed, however, in some examples the fourth orifice 316 may be plugged by the stem plug 322 of the interface member 302 (e.g., as depicted in FIG. 4. In some examples, the distal end of the lumen 308 may be enclosed or sealed by a portion of the valve stem 304 and/or a plug member. In additional or alternative configurations, the distal end of the lumen 308 may be open to the valve well and form a further orifice.

As discussed, the valve stem 304 may have one or more centering members 307. In some example configurations, the centering members 307 may facilitate proper alignment of the valve 300 within a valve well. Additionally or alternatively, one or more centering members 307 may be used to facilitate an overmolding procedure for forming a valve stem assembly. For example, the seals 306 and/or seal bodies 330 may be formed with an overmolding procedure and the centering members 307 may be used during the overmolding procedure to position the valve stem 304 and/or to retain flow during the overmolding procedure from running proximal of or distal of the centering members 307.

The centering members 307 of the valve stem 304 may have any suitable diameter. In some examples, a first (e.g., proximal) centering member 307a may have a first maximum outer diameter and a second (e.g., distal) centering member 307b may have a second maximum outer diameter that is different than or the same as the first diameter. As depicted in FIG. 6, the first centering member 307a may have a first maximum outer diameter that is greater than a second maximum outer diameter of the second centering member 307b, but other suitable configurations are contemplated.

As discussed with respect to FIG. 5, the seals 306 may be formed as part of one or more seal bodies 330. In some examples, when the first seal body 330a and the second seal body 330b are utilized, the second seal body 330b may be spaced from and distal of the first seal body 330a. In some examples, the first seal body 330a may be at least partially or entirely proximal of the second orifice 312 and the second seal body 330b may be at least partially or entirely distal of the second orifice 312. In some examples, the outer surface of the valve stem 304 proximate and/or around the second orifice 312 may be exposed between the first seal body 330a and the second seal body 330b, but other suitable configurations are contemplated. In some examples, the second seal body 330b may terminate proximal of the distal end of the valve stem 304. Terminating proximal of the distal end of the valve stem 304 such that the second seal body 330b does not extend around the distal end of the valve stem 304 may facilitate allowing the valve stem 304 to traverse a desired distance within the valve well and/or may provide other desired benefits. In alternative or additional configurations, it is contemplated the second seal body 330b may extend around the distal end of the valve stem 304.

The seals 306 may have any suitable maximum outer diameters. In some examples, the maximum outer diameters of one or more of the seals 306 or all of the seals 306 may be greater than an outer diameter of at least a portion of an associated seal body 330 between two consecutive, axially spaced seals 306. In some examples, the seals 306 may all have the same maximum outer diameters. In some examples, one or more of the seals 306 may have a maximum outer diameter that is different than a maximum outer diameter of at least one other seal 306. In some examples and as depicted in FIG. 6, the first seal 306a may have a first maximum outer diameter D1, the second seal 306b may have a second maximum outer diameter D2, the third seal 306c may have a third maximum outer diameter D3, the fourth seal 306d may have a fourth maximum outer diameter D4, the fifth seal 306e may have fifth maximum outer diameter D5, and the sixth seal 306f may have a sixth maximum outer diameter D6.

The seals of the first seal body 330a may have any suitable diameters. In one example configuration, the second maximum outer diameter D2 of the second seal 306b may be less than the first maximum outer diameter D1 of the first seal 306a. When the third seal 306c is included, the third maximum outer diameter D3 may be less than one or both of the first maximum outer diameter D1 and the second maximum outer diameter D2. In one example, the second maximum outer diameter D2 may be less than the first maximum outer diameter D1 and the third maximum outer diameter D3 may be less than the second maximum outer diameter D2, but other suitable configurations are contemplated.

The seals of the second seal body 330b may have any suitable diameters. In one example configuration, the fifth maximum outer diameter D5 of the fifth seal 306e may be less than the fourth maximum outer diameter D4 of the fourth seal 306d. When the sixth seal 306f is included, the fourth maximum outer diameter D4 may be greater than one or both of the fifth maximum outer diameter D5 and the sixth maximum outer diameter D6. In some examples, the fifth maximum outer diameter D5 and the sixth maximum outer diameter D6 may be equal to one another, as depicted for example in FIG. 6, but other suitable configurations are contemplated.

As discussed, the seals 306 may have any suitable configuration including, but not limited to a wiper seal confirmation. In some examples, one or more of the seals 306 may include a thin portion or wiper portion that deforms when introduced into a valve well. In some examples, one or more of the seals 306 may form a slight interference fit with the valve well. In one example, a wiper portion of the seal 306 may deflect proximally while also compressing radially inward such that the seal 306 may slide in the distal direction into a lumen or channel of the valve well and keep contact with the valve well to prevent fluid from passing longitudinally past the seal in either of the proximal direction or the distal direction. In various configurations, the seals 306 may be used to accommodate desired manufacturing tolerances, such as by allowing a wide range of diameters with consistent friction for sealing along an inner wall of the valve well.

As discussed, the valve stem 304 and/or the seals 306 may be formed from any suitable type of material. Example suitable materials for the valve stem 304 and/or the seals 306 may include, but is not limited to, metal materials, polymer materials, plastic materials, bioplastic materials, recyclable materials, thermoplastic materials, lubricious materials, flexible materials, thermoplastic elastomers (TPE), thermoplastic polyurethane (TPU), polycarbonate, acrylonitrile butadiene styrene (ABS), high-density polyethylene (HDPE), nylon, polyether ether ketone (PEEK), silicone, liquid silicone rubber (LSR), steel, aluminum, and/or other suitable material.

A thin wall geometry for one or more of the seals 306 may allow for a rigid material, such as HPDE, to be used for the seal 306, which can also facilitate formation of the seal 306 from the same material (e.g., HDPE) as the valve stem 304. In some examples, a wall of the valve stem 304 defining the lumen 308 may be sufficiently thick to provide adequate rigidity and strength to the monolithic stem during use while a thinner wall of at least a portion of one or more of the seals 306 may provide adequate flexibility for the seals 306.

One or more components of a valve stem assembly including, but not limited to, the valve stem 304, the seals 306, the centering members 307 the lumen 308, the seating members 309, and the orifices 310, 312, 314, 316 may be integrally formed as a unitary structure. In other words, in one or more configurations, the valve stem assembly may include the valve stem 304 and one or more additional components that are formed as a unitary structure.

In many examples described herein, the use of “monolithic stem” may include the valve stem 304 that is integrally formed with at least one seal and/or at least one flange, regardless of whether other seals and/or flanges are integrally formed with the valve stem 304. However, one or more of the stem-forming and valve assembly features described herein (e.g., a forming core pin that is removable via the fourth orifice 316 at a proximal end of the valve stem 304, and then plugged with the stem plug 322 of the interface member 302) may be utilized with valve stems 304 that are not monolithic valve stems (e.g., a valve stem assembled from two pieces), or with valve stems 304 that may be partially monolithic (e.g., a first portion of the valve stem 304 is integrally formed with one or more seals 306 and/or flanges, and one or more seals 306 and/or flanges for a second portion of the valve stem 304 are not integrally formed with the second portion of the valve stem 304). In one example, only the valve stem 304 itself may be monolithically formed. Accordingly, in some examples, the valve stem 304 may not be integrally formed with any seals 306 and/or flanges.

In various embodiments, some components of the valve 300 may be assembled onto, overmolded, and/or formed via additional processing of the valve stem 304. In some examples, one or more of the seals 306 may be overmolded and/or assembled onto the valve stem 304. For example, one or more of the seals 306 may be formed from a material the same as, different than, or separate from one or more other material (e.g., a material of the valve stem 304) and then assembled onto the valve stem 304, or overmolded directly onto the valve stem 304.

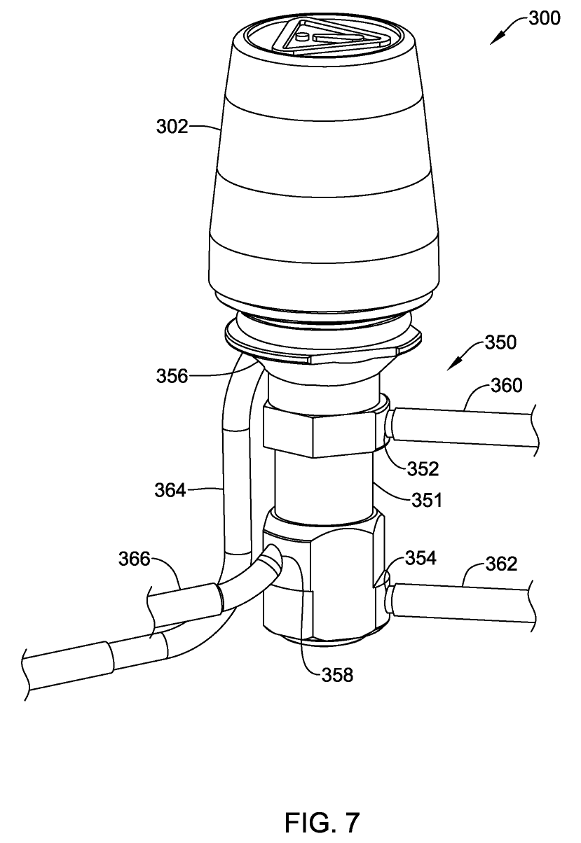

FIG. 7 schematically depicts a perspective view of the valve 300 positioned within a valve well 350 of an endoscope (e.g., the endoscope 100 and/or other suitable endoscopes). As depicted in FIG. 7, the interface member 302 of the valve may extend over and couple with at least a portion of the valve well 350 when the valve 300 is inserted into the valve well 350.

The valve well 350 may have any suitable configuration. In some examples, the valve well 350 may be the same as, may include features of, and/or may be different than the valve locations 135 discussed herein. As depicted in FIG. 7, the valve well 350 may include a valve well body 351 having a gas inlet 352 (e.g., an air inlet) configured to couple with a gas inlet tubing 360, a liquid (e.g., water) inlet 354 configured to couple with liquid inlet tubing 362, a gas outlet 356 configured to couple with gas outlet tubing 364, and a liquid outlet 358 configured to couple with liquid outlet tubing 366, but other suitable configurations are contemplated.

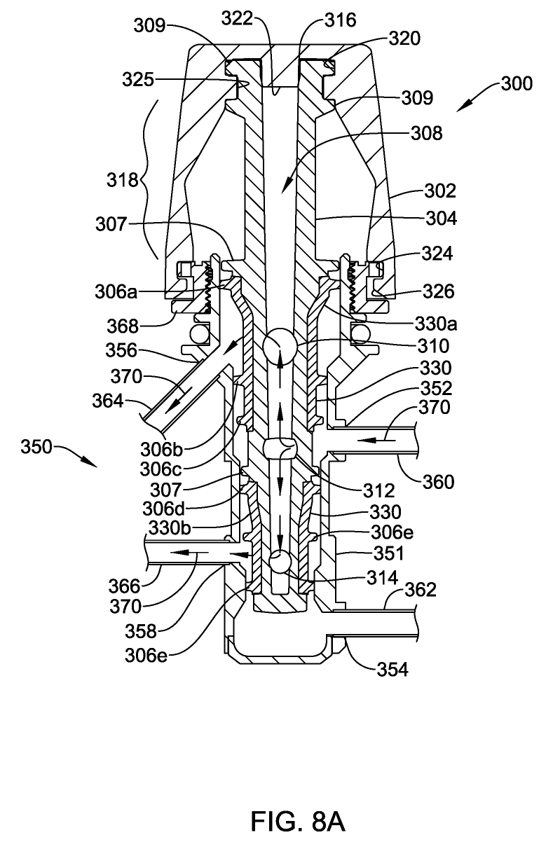

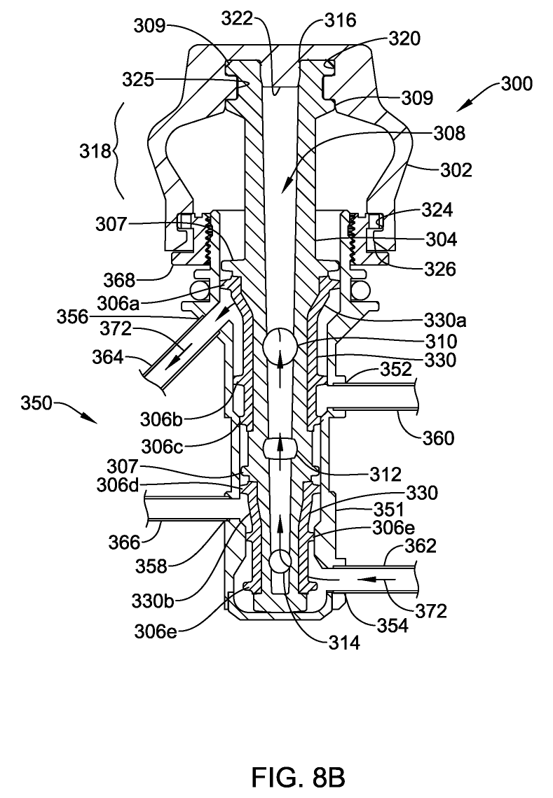

FIGS. 8A and 8B schematically depict cross-section views of the valve 300 within the valve well 350. FIG. 8A schematically depicts the valve 300 in the first configuration within the valve well 350. FIG. 8B schematically depicts the valve 300 in the second configuration within the valve well 350. In some examples, the first configuration may include a standby state (e.g., loaded into valve well 350, but not depressed by a user) while the second configuration may include a flushing state (e.g., loaded into valve well 350 and depressed by a user). Although the configuration of the valve 300 depicted in FIGS. 8A and 8B are the same as or similar to the configurations described herein, other suitable configurations of the valve 300 may be utilized and adjusted between the first configuration and the second configuration.

As discussed, FIGS. 8A and 8B include the valve 300 positioned within the valve well 350. As depicted in FIG. 8A, the valve 300 is configured or positioned with the spring portion 318 of the interface member 302 in an unactuated position, which may correspond to the first configuration of the valve. As depicted in FIG. 8B, the valve 300 is configured or positioned with the spring portion 318 of the interface member 302 in an actuated position, which may correspond to the second configuration of the valve 300 and advancing the valve stem 304 in a distal direction.

As depicted in FIGS. 8A and 8B, the valve well 350 may include a connection flange 368 configured to engage the well recess 324 of the interface member 302 to couple the interface member 302 with the valve well 350. In some examples, the connection flange 368 may include a circumferential protrusion that may be inserted into the well recess 324 of the interface member 302 to facilitate coupling the valve 300 with the valve well 350, but other suitable configurations are contemplated.

As discussed, the valve well 350 may include the gas inlet 352 coupled with the gas inlet tubing 360, the liquid inlet 354 coupled with the liquid inlet tubing 362, the gas outlet 356 coupled with the gas outlet tubing 364, and the liquid outlet 358 coupled with the liquid outlet tubing 366. Although FIGS. 8A and 8B depict the gas inlet 352 and the liquid inlet 354 on a same side of the valve well 350 (e.g., at different axial locations, but the same circumferential or radial location) and located axially between the gas outlet 356 and the liquid outlet 358, and the gas outlet 356 and the liquid outlet 358 on a same side of the valve well 350 (e.g., at different axial locations, but the same circumferential or radial location), the gas inlet 352, the liquid inlet 354, the gas outlet 356, and the liquid outlet 358 may be at or more other suitable axial, circumferential, or radial locations along the valve well 350.

In the first configuration (e.g., the standby state) of the valve 300, the valve 300 may be loaded into and coupled with the valve well 350, but not depressed or actuated (e.g., by a user). In some examples, the interface member 302 may snap and/or engage with the valve well 350 to couple the valve 300 with the valve well 350 and hold the valve 300 in the valve well 350 against a positive system pressure. When in the first configuration, a pump may be pumping a fluid (e.g., air or CO2) into the gas inlet 352 and through the valve 300 and as such, securing the interface member 302 with the valve well 350 may maintain the valve 300 in a desired position within the valve well 350 while fluid is pumped through the valve 300 and the valve well 350.

Fluid may flow through the valve 300 as depicted, for example, by the flow arrows 370 when the valve is in the first configuration, as depicted in FIG. 8A. For example, the fluid may pass through the gas inlet tubing 360, into the gas inlet 352, and into the valve well 350. The second seal 306b and the fourth seal 306d may engage an inner wall of the valve well 350 and prevent the fluid from moving within the valve well proximal of the second seal 306b or distal of the fourth seal 306d, which may result in the fluid flowing into the second orifice 312 and the lumen 308 of the valve stem 304. Once in the lumen 308, the fluid may flow out of the first orifice 310 into the valve well 350 and out of the gas outlet 356 into the gas outlet tubing 364. Additionally, once in the lumen 308, the fluid may flow out of the third orifice 314 into the valve well 350 and out of the liquid outlet 358 into the liquid outlet tubing 366. Once the fluid enters the valve well 350 between the first orifice 310 and the gas outlet 356, the fluid may be directed out of the gas outlet 356 due to the first seal 306a engaging the inner wall of the valve well 350 and the second seal 306b engaging the inner wall of the valve well 350, as depicted for example in FIG. 8A. Once the fluid enters the valve well 350 between the third orifice 314 and the liquid outlet 358, the fluid may be directed out of the liquid outlet 358 due to the fourth seal 306d engaging the inner wall of the valve well 350 and the sixth seal 306f engaging the inner wall of the valve well 350, as depicted for example in FIG. 8A. Further, in the first configuration of the valve 300, fluid from the liquid inlet 354 may be blocked from entering the valve stem 304 via the gas inlet 352 due to the sixth seal 306f engaging the inner wall of the valve well 350 proximal of the liquid inlet 354.

In the second configuration (e.g., the flushing state), the valve 300 may be depressed by a user such that the spring portion 318 of the interface member 302 is axially compressed and the valve stem 304 may be advanced in a distal direction within the valve well 350, as depicted for example in FIG. 8B. No flow from the gas inlet 352 is provided to the lumen 308 of the valve stem 304 when valve 300 is in the second configuration due to the second seal 306b engaging the inner wall of the valve well 350 at a location proximal of the gas inlet 352 and the third seal 306c engaging the inner wall of the valve well 350 at a location distal of the gas inlet 352 such that flow from the gas inlet 352 is blocked from the orifices 310, 312, 314, 316 in communication with the lumen 308, the liquid inlet 354, the gas outlet 356, and the liquid outlet 358, as depicted for example in FIG. 8B.

Fluid (e.g., liquid, such as water) may flow through the valve 300 as depicted, for example, by the flow arrows 372 when the valve is in the second configuration, as depicted in FIG. 8B. For example, the fluid may pass through the liquid inlet tubing 362, into the liquid inlet 354, and into the valve well 350 distal of the fifth seal 306e, which may be engaging the inner wall of the valve well 350. The fifth seal 306e engaging the inner wall of the valve well 350 may prevent the fluid from moving within the valve well proximal of the fifth seal 306e, which may result in the fluid

flowing into the third orifice 314 and the lumen 308 of the valve stem 304. Once in the lumen 308, the fluid may flow out of the first orifice 310 into the valve well 350 and out of the gas outlet 356 into the gas outlet tubing 364 to flush the gas outlet tubing 364. Once the fluid enters the valve well 350 between the first orifice 310 and the gas outlet 356, the fluid may be directed out of the gas outlet 356 due to the first seal 306a engaging the inner wall of the valve well 350 and the second seal 306b engaging the inner wall of the valve well 350, as depicted for example in FIG. 8B. Further, in the second configuration of valve 300, the liquid outlet 358 may be blocked from receiving fluid entering the valve well 350 or the valve stem 304 via the liquid inlet 354 due to the fourth seal 306d engaging the inner wall of the valve well 350 proximal of the liquid outlet 358 and the fifth seal 306e engaging the inner wall of the valve well 350 distal of the liquid outlet 358, as depicted for example in FIG. 8B.

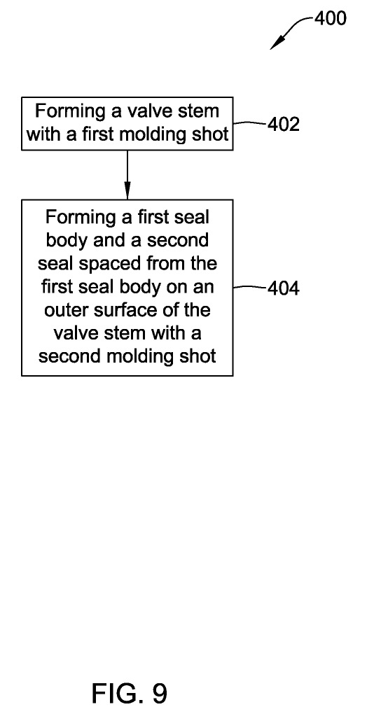

FIG. 9 depicts a schematic box diagram of an illustrative method 400 of forming a valve. The valve formed using the method 400 may have features of the valve 300 described herein and/or other suitable features.

The method 400 may include forming 402 a valve stem with a first molding shot. For example, the valve stem may be formed with an injection molding process that includes injecting a first material into a mold having a three-dimensional shape of a desired valve stem. In some examples, the three-dimensional shape of the desired valve stem may be configured to form a valve stem having a proximal end, a distal end, an outer surface, a first orifice, a second orifice, a third orifice, a fourth orifice, and a lumen in fluid communication with the first orifice, the second orifice, the third orifice, and the fourth orifice. The three-dimensional shape of the mold may be configured to form a valve stem with additional or alternative features or components, as described herein or otherwise.

When forming 402 the valve stem with the first molding shot, a forming core pin may be utilized. For example, the lumen of the valve stem may be created with the forming core pin (or core pin) during the first molding shot by forming the valve stem around the forming core pin extending in an axial direction of the valve stem. After the first molding shot, the forming core pin may be removed from the vale stem via a formed axial orifice of the valve stem to define the lumen of the valve stem. Additionally, the forming core pin, when utilized, may be used as a support and/or manipulation member as other components of the valve stem are formed and/or may be used for other suitable purposes.

The method 400 may include forming 404 a first seal body and a second seal body on the outer surface of the valve stem with a second molding shot. For example, the first seal body and the second seal body may be formed through an overmold process that includes injecting a second material into a mold having a three-dimensional shape of a desired valve stem assembly comprising the valve stem (previously molded or formed), the first seal body, and the second seal body. In some examples, the three-dimensional shape of the desired valve stem assembly may be configured to form the first valve body on the outer surface of the valve stem with one or more first openings aligned with the first orifice of the valve stem and a plurality of seals and the second valve body on the outer surface of the valve stem and axially spaced from the first seal body, where the second seal body may include one or more second openings aligned with the third orifice of the valve stem and a plurality of seals. The first valve body may include a first seal proximal of the one or more first openings, a second seal distal of one or more first openings, and a third seal distal of the second seal and the second valve body may include a fourth seal proximal of the one or more second openings, a fifth seal distal of the fourth seal and proximal of the one or more second openings, and a sixth seal distal of the one or more second openings. Other suitable configurations of the seals of the first seal body and the second seal body are contemplated. Although the method 400 pf forming the valve stem assembly is described as comprising two molding shots, it is contemplated that the valve stem assembly may be formed from a single molding shot with a single material (e.g., to form a monolithic, integrally formed valve stem assembly) and/or more than two molding shots.

Once the valve stem assembly has been formed, an interface member may be coupled with the valve stem assembly. In some examples, a stem plug of the interface member may be inserted into the fourth orifice of the valve stem to seal the fourth orifice. Additionally or alternatively, the orifice member may couple with the valve stem in one or more other suitable manners including, but not limited to, a snap fit, a snap interference fit, a threaded fit, and/or one or more other suitable coupling techniques.

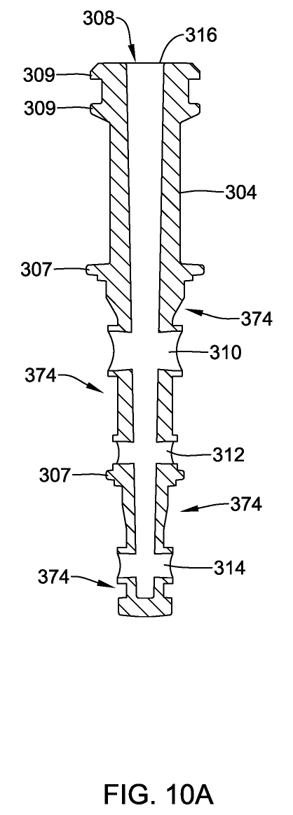

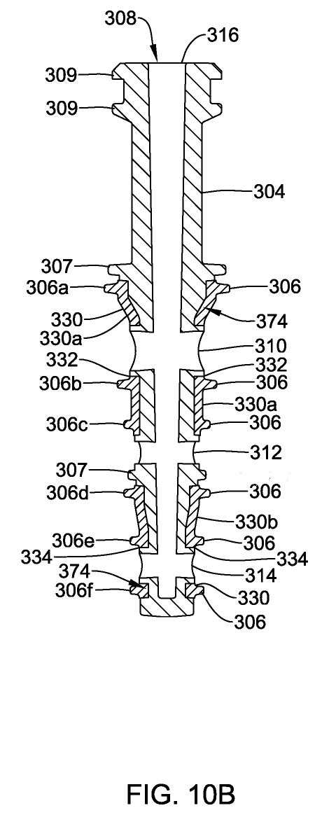

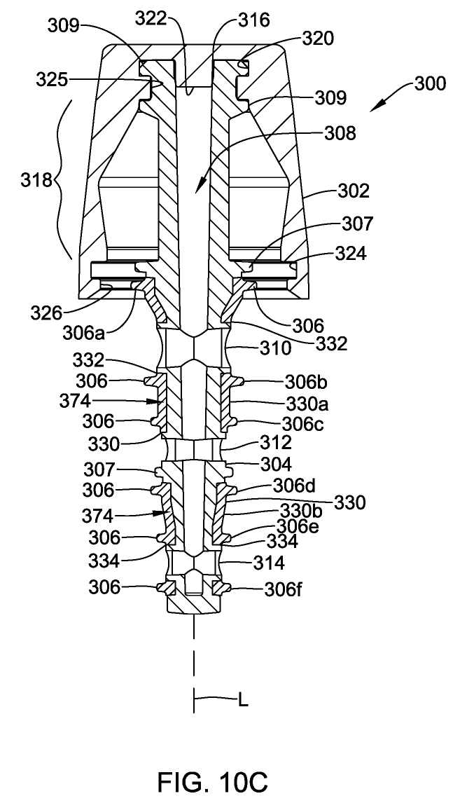

FIGS. 10A-10C schematically depict steps of assembling the valve 300. FIG. 10A depicts a schematic cross-section view of the valve stem 304. In some examples, the valve stem 304 may be formed using an injection molding process with a first shot of material into a mold, but other suitable forming techniques (e.g., machining techniques, finishing techniques, etc.) may be utilized. The valve stem 304 may include, among additional or alternative features, the proximal end, the distal end, an outer surface, centering members 307, the lumen 308, the seating members 309, the first orifice 310, the second orifice 312, the third orifice 314, the fourth orifice 316, and/or other suitable features. In some examples, the valve stem 304 may include one or more seal recesses 374 configured to receive material that is used to form the seals 306 and/or the seal bodies 330 during a molding process.

The seals 306 and/or the seal bodies 330 may be overmolded onto the valve stem 304, as depicted in FIG. 10B, and/or formed on the valve stem 304 or applied to the valve stem 304 in one or more other suitable manners. In some examples, the seal bodies 330 may be formed or located within the seal recesses 374. As depicted in FIG. 10B, the first seal body 330a and the second seal body 330b may be overmolded onto the outer surface of the valve stem 304 such that the first seal body 330a is proximal of the second orifice 312 and the one or more first openings 332 of the first seal body 330a are aligned with the first orifice 310 and the second seal body 330b is axially spaced from the first seal body 330a, distal of the second orifice 312, and the one or more second openings 334 of the second seal body 330b is aligned with the third orifice 314.

Once the valve stem assembly has been formed, the interface member 302 may be coupled with the proximal end of the valve stem 304, as depicted for example in FIG. 10C. In some examples, the stem plug 322 of the valve interface member 302 may be inserted into the fourth orifice 316 of the valve stem 304 to seal the fourth orifice 316. Additionally or alternatively, the interface member 302 may couple with the valve stem 304 in one or more other suitable manners including, but not limited to, a snap fit, a snap interference fit, a threaded fit, and/or one or more other suitable coupling techniques. In one example of a snap fit or snap interference fit coupling, the stem protrusion 325 may be inserted into a recess of the valve stem 304 between the seating members 309 and the seating members 309 may be inserted into the stem recesses 320 of the interface member 302. Other suitable assembly techniques of the valve 300 are contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1. A valve for a medical device, comprising:
a valve stem including a proximal end, a distal end, an outer surface, a first orifice, a second orifice distal of the first orifice, and a lumen in fluid communication with the first orifice and the second orifice; and
a first seal body extending along the outer surface of the valve stem, the first seal body having an opening aligned with the first orifice, a first seal proximal of the opening, and a second seal distal of the opening.

2. The valve of claim 1, wherein the valve stem and the first seal body are formed from one or more polymers.

3. The valve of claim 1, wherein the first seal has a first maximum outer diameter and the second seal has a second maximum outer diameter that is less than the first maximum outer diameter.

4. The valve of claim 1, wherein the first seal body comprises a third seal distal of the second seal.

5. The valve of claim 4, wherein the first seal has a first maximum outer diameter, the second seal has a second maximum outer diameter, and the third seal has a third maximum outer diameter that is less than one or both of the first maximum outer diameter and the second maximum outer diameter.

6. The valve of claim 1, further comprising:
a second seal body extending along the outer surface of the valve stem spaced from and distal of the first seal body.

7. The valve of claim 6, wherein the second seal body terminates proximal of the distal end of the valve stem.

8. The valve of claim 6, wherein the second seal body comprises an opening aligned with the second orifice.

9. The valve of claim 8, wherein the second seal body comprises a first seal, a second seal distal of the first seal and proximal of the opening of the second seal body, and a third seal distal of the opening of the second seal body.

10. The valve of claim 6, wherein the second seal body comprises a first seal having a first maximum outer diameter, a second seal distal of the first seal and having a second maximum outer diameter, and a third seal distal of the second seal and having a third maximum outer diameter, wherein the first maximum outer diameter is greater than one or both of the second maximum outer diameter and the third maximum outer diameter.

11. The valve of claim 10, wherein the second maximum outer diameter and the third maximum outer diameter are equal.

12. A valve for a medical device, comprising:
an interface member;
a valve stem configured to couple with the interface member, including a proximal end, a distal end, an outer surface, a first orifice, a second orifice distal of the first orifice, and a lumen in fluid communication with the first orifice and the second orifice;
a first seal body extending along the outer surface of the valve stem, the first seal body having an opening aligned with the first orifice and a plurality of seals; and
a second seal body extending along the outer surface of the valve stem and spaced from the first seal body, the first seal body having an opening aligned with the second orifice and a plurality of seals.

13. The valve of claim 12, wherein the proximal end of the valve stem is open to the lumen and the distal end of the valve stem is closed to the lumen.

14. The valve of claim 13, wherein the interface member is configured to extend through the open proximal end and into the lumen of the valve stem.

15. The valve of claim 12, wherein the plurality of seals of the first seal body comprises at least two seals distal of the opening of the first seal body and the plurality of seals of the second seal body comprises at least two seals proximal of the opening of the second seal body.

16. The valve of claim 12, wherein the valve stem comprises a third orifice in fluid communication with the lumen and located between the first orifice and the second orifice.

17. The valve of claim 12, wherein the valve stem comprises a third orifice in fluid communication with the lumen and located between and spaced from the first seal body and the second seal body.

18. A method of manufacturing a valve for a medical device, the method comprising:
forming a valve stem with a first molding shot, the valve stem including a proximal end, a distal end, an outer surface, a first orifice, a second orifice distal of the first orifice, and a lumen in fluid communication with the first orifice and the second orifice; and
forming a first seal body and a second seal body spaced from the first seal body on the outer surface of the valve stem with a second molding shot.

19. The method of claim 18, further comprising:
sealing a proximal opening of the valve stem with an interface member.

20. The method of claim 18, wherein the first seal body comprises an opening aligned with the first orifice and a plurality of seals, and the second seal body comprises an opening aligned with the second orifice and a plurality of seals.
